(19) United States
(12) Patent Application Publication
(10) Pub. No.: US 2023/0217875 A1
(43) Pub. Date: Jul. 13, 2023

(54) IN VITRO EMBRYO RESCUE

(71) Applicants: Claudia KOHLER, Uppsala (SE); Wenjia XU, Uppsala (SE)

(72) Inventors: Claudia KOHLER, Uppsala (SE); Wenjia XU, Uppsala (SE)

(21) Appl. No.: 18/009,242

(22) PCT Filed: Mar. 25, 2021
(86) PCT No.: PCT/SE2021/050264

§ 371 Date: Dec. 8, 2022

(30) Foreign Application Priority Data
Jun. 9, 2020 (SE) 2050671-3

(51) Int. Cl. A01H 4/00; A01H 1/02; C12N 5/00

(52) U.S. Cl. CPC A01H 4/002; A01H 1/021; C12N 5/0025; C12N 2500/34

(57) ABSTRACT

The present invention relates to the use of trehalose, and/or a derivative thereof, as a supplement to a culture medium for culturing hybrid plant embryos in in vitro embryo rescue. The addition of trehalose, and/or a derivative thereof, to the culture medium significantly increased the survival rate of the hybrid plant embryos.

### TECHNICAL FIELD

The present invention generally relates to the field of plant cross-breeding, and in particular to in vitro embryo rescue of hybrid plant embryos.

### BACKGROUND

Plant cross-breeding is the purposeful manipulation of plant species in order to take advantage of heterosis and genetic variation, create desired genotypes and phenotypes for specific purposes, such as increasing yield and quality, resistance to biotic and abiotic stress, and/or to generate novel traits of high economic value, e.g., seedless fruits, such as banana, watermelon and citrus. Interspecies and interploidy hybridizations are two breeding strategies frequently applied for crop improvement. Interspecies or interspecific hybridization is the most widely used strategy for broadening the genetic variability of target traits. Many agriculturally valuable traits have been transferred from wild species or cultivated species to other cultivated species. Interploidy hybrids and, in particular triploid plants, have also great economic value. Fruit breeding programs have great interest in combining desirable genetic traits of complementary parents at the triploid level for the purpose of developing improved seedless fruits. Two of the most famous examples are cultivated banana, a natural triploid plant species, and seedless watermelon, an artificial breeding product, because of their high quality flesh that is virtually free of seeds. Similar applications are also utilized in other seedless fruit, such as citrus and apple.

However, interploidy and interspecies hybridizations rarely yield viable seeds, due to a post-fertilization incompatibility barrier in the endosperm, resulting in seed abortion. In interploidy hybridizations in Arabidopsis thaliana, e.g. diploid maternal×tetraploid paternal crosses, or interspecific hybrids in Capsella, the development of hybrid embryos starts arresting around the torpedo stage (Kradolfer et al. 2013; Rebernig et al. 2015) and the endosperm degenerates due to the failure of cellularization, finally resulting in shrunken seeds, which fail to germinate. This phenomenon has been termed “triploid block” (Marks, 1966; Esen & Soost 1973; Köhler et al. 2010), which seriously hinders the application of cross-breeding. Therefore, overcoming these postzygotic reproductive bottlenecks will be of great value for plant cross-breeding.

In vitro embryo rescue is an efficient and indispensable plant breeding tool to protect embryos from premature abortion, allowing generation of viable interspecies and interploidy hybrids (Sharma 1995). For example, fruit breeding programs have greatly increased interest in exploiting interploidy hybridizations to develop improved seedless fruits. However, success of this approach has only been reported in a limited number of species due to various crossing barriers and embryo abortion at early stages. The technique of immature embryo rescue overcomes seed abortion that occurs through abnormal endosperm development by surgically excising the immature embryo and germinating or culturing it in artificial media, independent of the endosperm. Thus, immature embryo rescue provides an alternative means to recover triploid hybrids, which usually fail to completely develop in vivo. However, the success rate using such techniques has nevertheless been low.

Sahijram et al. 2013 describes application of hybrid embryo rescue in production of intergeneric and interploidy plant hybrids in fruit crops, vegetable crops and ornamental crops.

Hence, there is a still a need for improvements within in vitro embryo rescue and, in particular, such in vitro embryo rescue of interploidy or interspecies hybrid embryos.

### SUMMARY

It is a general objective to provide an improved in vitro embryo rescue method.

It is a particular objective to increase the survival rate of hybrid plant embryos in an in vitro embryo rescue method.

These and other objectives are met by embodiments as disclosed herein.

The present invention is defined in the independent claims. Further embodiments are defined in the dependent claims.

An aspect of the invention relates to a method of in vitro embryo rescue. The method comprises culturing a hybrid plant embryo in a culture medium comprising trehalose, and/or a derivative thereof.

Another aspect of the invention relates to use of trehalose, and/or a derivative thereof, as a supplement to a culture medium for in vitro embryo rescue. In this aspect, a hybrid plant embryo is cultured in the culture medium supplemented with trehalose, and/or the derivative thereof.

A further aspect of the invention relates to an in vitro embryo rescue culturing system. The system comprises a culture vessel comprising a culture medium comprising trehalose, and/or a derivative thereof. The system also comprises a hybrid plant embryo present in the culture medium comprising trehalose, and/or the derivative thereof.

The present invention significantly increases the survival rate of rescued hybrid plant embryos, such as interploidy or interspecies hybrid plant embryos, in in vitro embryo rescue. A problem with prior art in vitro embryo rescue has been the low success rate, mainly due to low survival rate when culturing the hybrid plant embryos. Addition of trehalose, and/or the derivative thereof, to the culture medium increased the survival rate up to 90% as compared to corresponding culture medium lacking trehalose. The present invention thereby provides a significant improvement in the generation of plant hybrids, such as for the production of new crop varieties or seedless plants.

### BRIEF DESCRIPTION OF THE DRAWINGS

The embodiments, together with further objects and advantages thereof, may best be understood by making reference to the following description taken together with the accompanying drawings, in which:

FIG. 1. Embryo rescue for interploidy hybridization (Col 2××Col 4×) using an in vitro culture system. (A) Percentage of surviving triploid embryos after 14 days of in vitro culture on culture medium containing glucose (Glu), galactose (Gal), sucrose (Suc), fructose (Fru) or trehalose (Tre). (B) Percentage of surviving triploid embryos after 14 days of in vitro growth on culture media containing different concentrations of trehalose. (C-H) Pictures showing the comparison of embryos grown on glucose, galactose, sucrose, fructose or trehalose for 4 weeks. Morphology of triploid embryo grown on control culture medium for 7 days (I), or culture medium comprising trehalose for 7 days (J) and 10 days (K). Triploid seedling grown into an adult plant on MS medium (L) or Nitsch medium (M) supplemented with 1.5% trehalose for 3 weeks.

FIG. 2. Embryo rescue of interspecies hybrids (Capsella rubella×C. grandiflora) using an in vitro culture system. (A) Percentage of surviving embryos after 14 days of in vitro growth on glucose, galactose, sucrose, fructose or trehalose. (B) Percentage of surviving embryos after 14 days of in vitro growth on culture media containing different concentrations of trehalose. (C-H) Pictures showing hybrid embryos grown on glucose, galactose, sucrose, fructose or trehalose for 4 weeks. Hybrid seedling grown into an adult plant on MS medium (I) or Nitsch medium (J) supplemented with 3% trehalose for 3 weeks.

FIG. 3. Embryo rescue for interploidy hybridization (Oryza sativa L. cv. Suitou nourin 8 2××4×) using an in vitro culture system. (A) Percentage of surviving triploid embryos after 14 days of in vitro culture on medium containing sucrose, and trehalose. (B, C, D, E) the comparison of embryos grown on sucrose or trehalose with sucrose for 5 days. Morphology of triploid embryo grown on trehalose with sucrose medium for 14 days (F).

### DETAILED DESCRIPTION

The present invention generally relates to the field of plant cross-breeding, and in particular to in vitro embryo rescue of hybrid plant embryos.

The generation of hybrid plants, such as interploidy or interspecies hybrids, frequently requires rescuing immature hybrid plant embryos and cultivating the hybrid plant embryos in vitro, since hybrid embryo development is impaired by the surrounding endosperm. However, prior art in vitro embryo rescue protocols have suffered from low efficiency, including low survival rate of the rescued hybrid plant embryos. The present invention is based on the unexpected finding that the addition of the sugar trehalose, and/or a derivative thereof, to the culture medium significantly increased the survival rate of rescued hybrid plant embryos. This increase in survival rate is specific for trehalose, and derivatives thereof, since other sugars, including glucose, galactose, sucrose and fructose did not significantly improve the survival rate of rescued plant hybrid embryos. In fact, experimental data as presented herein showed that addition of trehalose in the culture medium could increase the survival rate of plant hybrid embryos with about 90% as compared to control, i.e., no sugar supplement, whereas the other sugars did not increase the survival rate at all or at most at up to 10% as compared to control.

The present invention can therefore find uses in the field of plant cross-breeding, specifically to interploidy and interspecies hybridizations, which are two common approaches for crop improvement, the production of new crop varieties and seedless plants. This invention allows development of immature plant embryos directly into viable hybrid plants.

Trehalose is a non-reducing disaccharide consisting of two glucose molecules linked by 1,1-glycosidic bonds. Although it exists widely in diverse organisms, its content in most plant species is extremely low. Trehalose is well-known for its accumulation to high concentrations in anhydrobiotic organisms that survive complete dehydration. In plants, trehalose is synthesized from uridine diphosphate glucose (UDPG) and 6-phospho-glucose (G6P) by trehalose-6-phosphate synthase (TPS) to form trehalose phosphate (Tre6P). The trehalose-6-phosphate phosphatase (TPP) dephosphorylates Tre6P to form trehalose and in the presence of trehalase, trehalose is degraded to glucose. Trehalose can serve as an energy source, osmolyte or protein/membrane protectant.

A derivative of threhalose as used herein refers to trehalose 6—phosphate (Tre6P), trehalose dihydrate, a trehalose-lipid, monoacyl α,α-trehalose, 3-ketotrehalose, D-trehalose-1,1′-d₂, trehalose 6,6′-dimycolate, α,β-trehalose, trehalose 6-phosphate dipotassium salt, trehalose 6-decanoate, trehalose 6-octanoate, trehalose 6-hexadecanoate, trehalose 6-tetradecanoate, and trehalose monooleate.

Interploidy hybridization is a hybridization or manual cross between two different individuals of different ploidy levels. Individuals resulting from this type of hybridization are called interploidy hybrids. An interspecies hybrid, also referred to as interspecific hybrid, is a cross between plants of two different species.

A hybrid plant embryo, such as an interploidy or interspecies hybrid embryo, as referred to herein is an embryo from sexual (interspecies or interploidy) fusion of male and female gametes. Hence, the hybrid plant embryo is a so-called zygotic embryo. Zygotic embryogenesis in plants is the developmental period, in which a zygote undergoes a series of differential events, leading to the formation of a mature embryo (zygotic embryo). This is in clear contrast to somatic embryogenesis, which is the process in which a somatic cell is induced to develop into an embryo (somatic embryo).

An aspect of the invention relates to a method of in vitro embryo rescue. The method comprises culturing a hybrid plant embryo in a culture medium comprising trehalose and/or a derivative thereof.

Trehalose, and/or the derivative thereof, is added as a supplement to the culture medium employed for hybrid plant embryo culturing and growth. The presence of trehalose, and/or the derivative thereof, in the culture medium significantly improves the survival rate of the hybrid plant embryo as compared to culturing the hybrid plant embryo in the culture medium lacking any trehalose, and/or the derivative thereof.

In an embodiment, the culture medium comprises trehalose. In another embodiment, the culture medium comprises a derivative of trehalose. It is also possible to use a culture medium supplemented with multiple different derivatives of trehalose or a culture medium comprising trehalose and at least one derivative of trehalose.

In an embodiment, the method comprises isolating the hybrid plant embryo from a seed of a plant. In such an embodiment, culturing the hybrid plant embryo comprises culturing the isolated hybrid plant embryo in the culture medium comprising trehalose, and/or the derivative thereof.

In a particular embodiment, the hybrid plant embryo is retrieved from a seed. The seed may in turn be obtained by, for instance, crossing plants of a given species but different ploidies in order to obtain interploidy seeds and hybrid embryos. For instance, a female gamete of a plant with a first ploidy can be fertilized with pollen from a plant with a second, different ploidy. An illustrative example is the production of triploid seeds by crossing of diploid and tetraploid plants. Another example is seeds obtained by crossing plants of different species, but optionally of the same genus, in order to get interspecies seeds. Hence, in an embodiment, the hybrid plant is isolated from an interploidy seed or from an interspecies seed.

A seed typically includes three main parts; a seed coat, an embryo and an endosperm that supplies nutrients to the embryo. In a particular embodiment, isolating the hybrid plant embryo from the seed comprises opening the seed coat and removing the hybrid plant embryo from the seed coat and the endosperm. Hence, hybrid plant embryo is preferably dissected from the seed and endosperm.

In an embodiment, isolating the hybrid plant embryo comprises dissecting the hybrid plant embryo from the seed in a culture medium. In a particular embodiment, this culture medium comprises trehalose, and/or the derivative thereof.

Generally, it is preferred to dissect the hybrid plant embryo directly into the culture medium, in which it should be cultured and grown. Firstly, hybrid plant embryos are very small and fragile and handling them is hard, in particular transferring them from a dissection area to a culturing or growth area. Hence, it is easier if the hybrid plant embryo once isolated or dissected from the seed is directly within the culture medium, in which it is to be grown or cultured. This culture medium then preferably comprises trehalose, and/or the derivative thereof, thereby relaxing the need for transferring the isolated hybrid plant embryo from one culture medium lacking trehalose, and/or the derivative thereof, to another culture medium comprising trehalose, and/or the derivative thereof. Secondly, dissecting the hybrid plant embryo from the seed in the culture medium reduces the risk for contamination of the hybrid plant embryo during the isolation process.

In an embodiment, isolating the hybrid plant embryo comprises opening a silique of the plant immersed in a culture medium to retrieve the seed. In a particular embodiment, this culture medium comprises trehalose, and/or the derivative thereof.

In this embodiment, the seed is contained in a silique or silique, i.e., a seed capsule. The seed is then dissected from this seed capsule and the opening of the silique and retrieval of the seed thereof preferably takes place while the silique is immersed in a culture medium, preferably comprising trehalose, and/or the derivative thereof. As mentioned above, such an approach simplifies handling of seeds and embryos and reduces the risk for contamination.

In an embodiment, culturing the hybrid plant embryo comprises culturing a hybrid plant embryo at a heart embryogenesis stage or a torpedo embryogenesis stage.

For some plants, such as Cruciferous species, seed development comprises a number of stages, I: zygote, II: proembryo, III: globular, IV: heart, V: torpedo and VI: mature embryo.

In many interspecies and interploidy crosses, hybrid plant embryos frequently abort in the developing seeds. Depending on the species, hybrid plant embryos fail to progress beyond the globular, heart or torpedo stage (Gadwal et al. 1968; Kradolfer et al. 2013; Rebernig et al. 2015; Zhang et al., 2016). Hence, in a preferred embodiment, the hybrid plant embryo is isolated from a seed that is at a stage prior to arrest, such as globular stage, the heart stage or the torpedo stage.

In an embodiment, culturing the hybrid plant embryo comprises culturing an interploidy hybrid plant embryo in the culture medium comprising trehalose, and/or the derivative thereof. Interploidy hybrid plant embryo is the plant embryo obtained from interploidy hybridization, i.e., the hybridization or crossing between different plant individuals of different ploidy levels. A diploid plant comprises two homologous copies of each chromosome, whereas a polyploid plant comprises more than two sets of chromosomes, such as three sets (triploid), four sets (tetroploid), five sets (pentaploid), and so on. A typical example of an interploidy hybrid plant embryo is a triploid hybrid plant embryo obtained by crossing of diploid and tetraploid plants.

In another embodiment, culturing the hybrid plant embryo comprises culturing an interspecies hybrid plant embryo in the culture medium comprising trehalose, and/or the derivative thereof. Interspecies hybrid plant embryo as used herein includes embryos obtained by crossing plants of different species.

The plants of different species are typically of the same genus but may be of different genera, sometimes referred to as intergeneric hybrids. Hence, interspecies hybrid plant embryo as used herein includes hybrid plant embryos obtained by crossing plants of different species or of different genera, preferably of different species but the same genus.

The hybrid plant embryo is a hybrid plant zygotic embryo formed by fusion of male and female gametes, preferably formed by interspecies or interploidy fusion of male and female gametes from plants of different species or of different ploidy levels.

The hybrid plant embryo could be a hybrid embryo from any plant including monocotyledons and dicotyledons.

In an embodiment, culturing the hybrid plant embryo comprises culturing the hybrid plant embryo in the culture medium comprising trehalose, and/or the derivative thereof, at a concentration of from 0.5% (w/v) up to 10% (w/v). In a particular embodiment, the culture medium comprises trehalose, and/or the derivative thereof, at a concentration of from 0.75% (w/v) up to 8% (w/v) or from 1% (w/v) up to 8% (w/v), and more preferably at a concentration of from 1.25% (w/v) up to 6% (w/v) or from 1.5% (w/v) up to 6% (w/v).

w/v as used herein indicates weight/volume percentage, such as in g/mL.

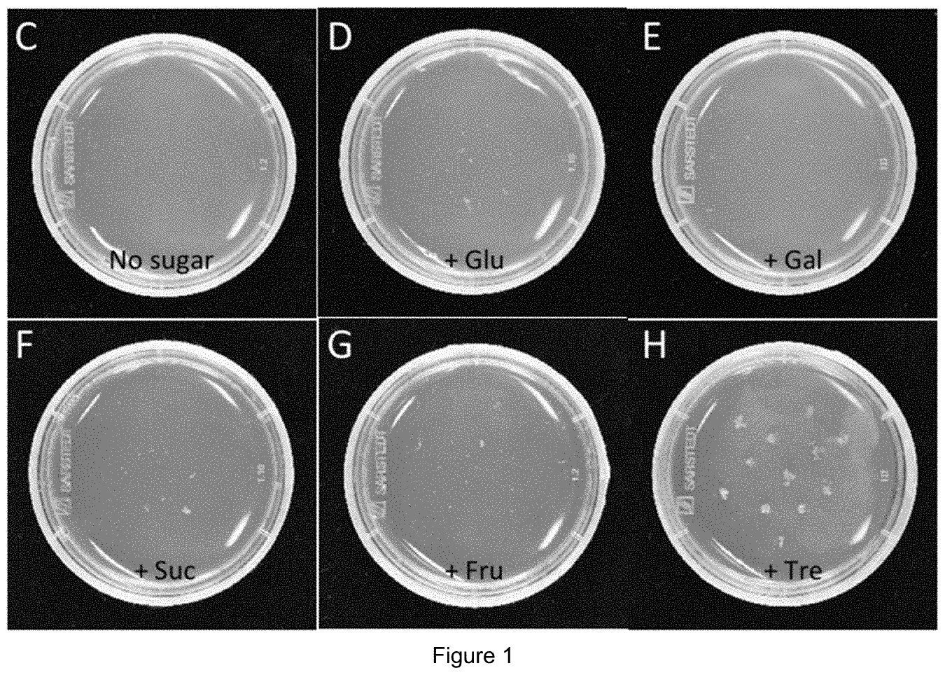

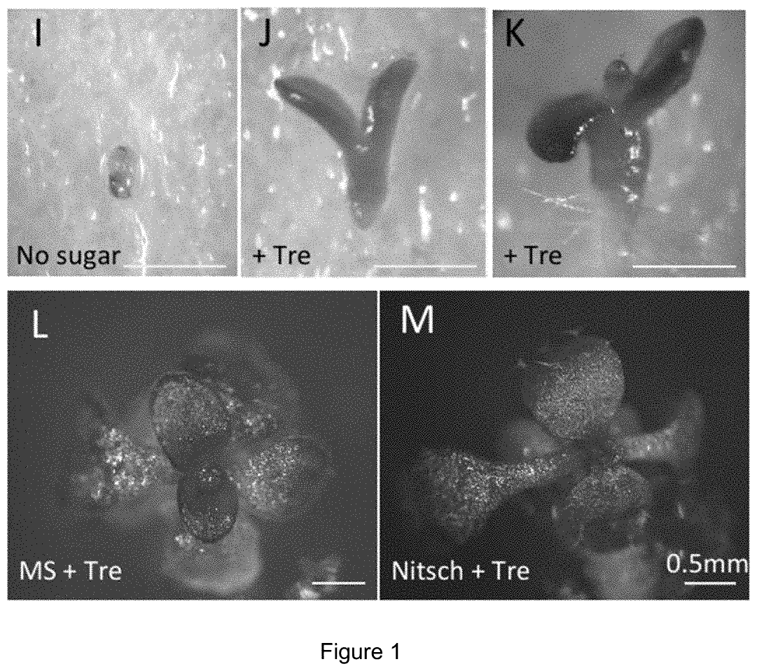

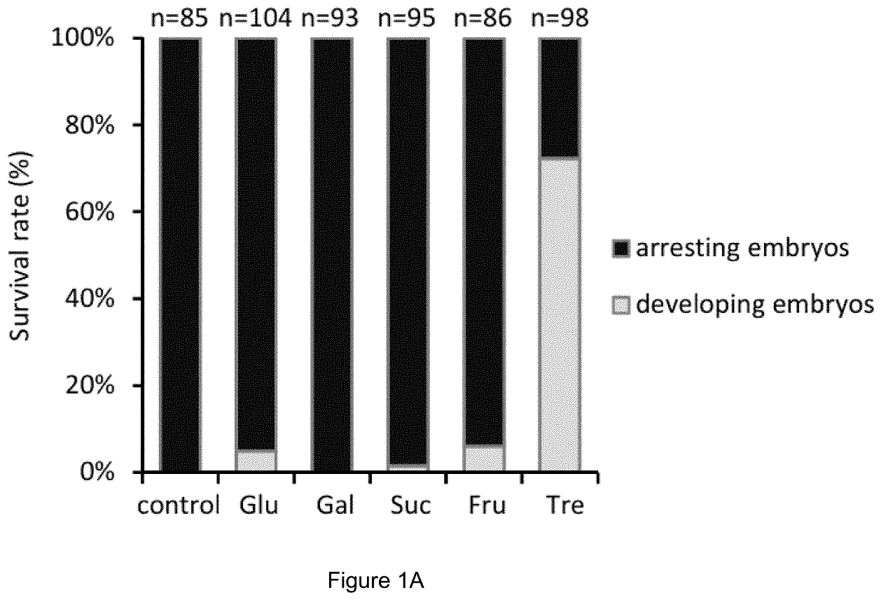

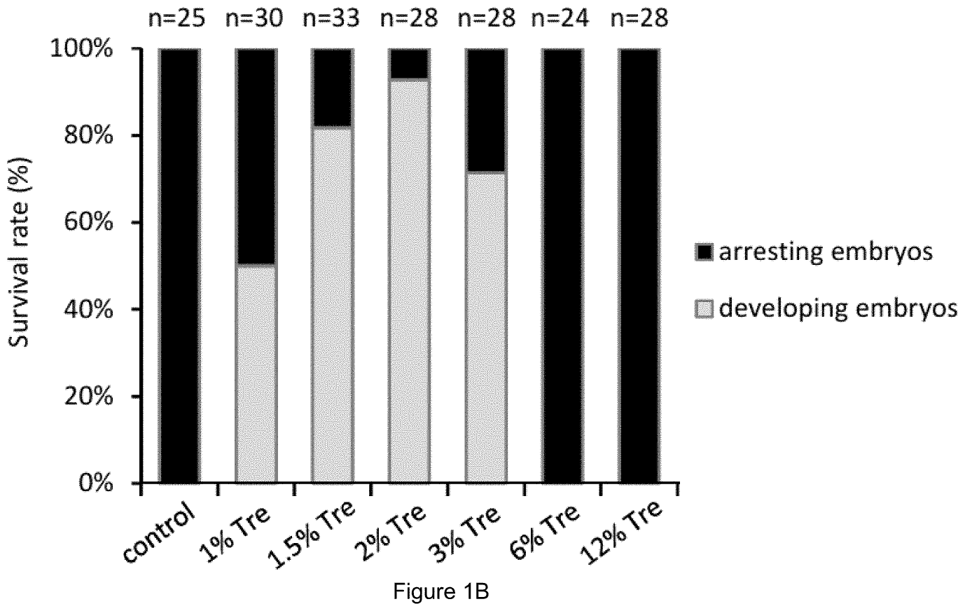

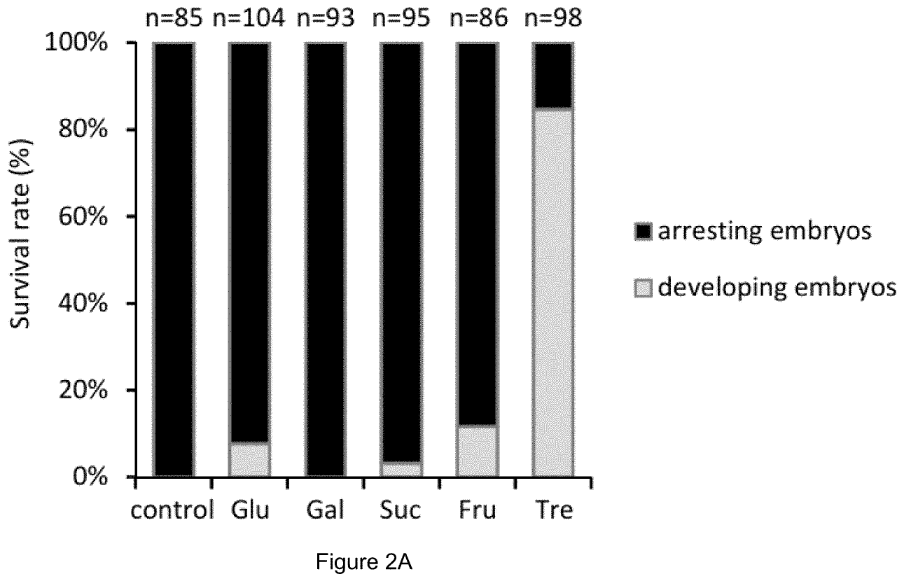

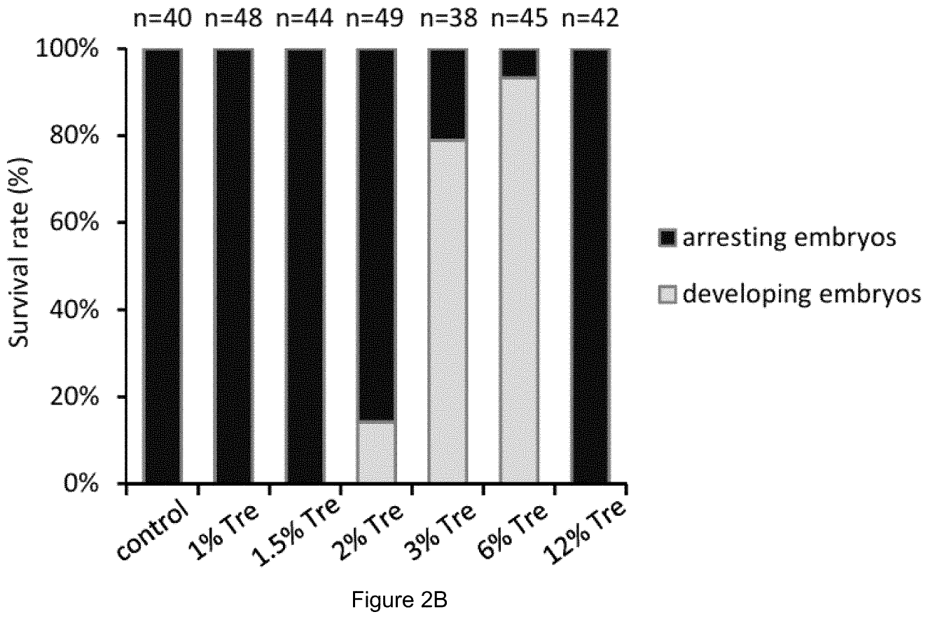

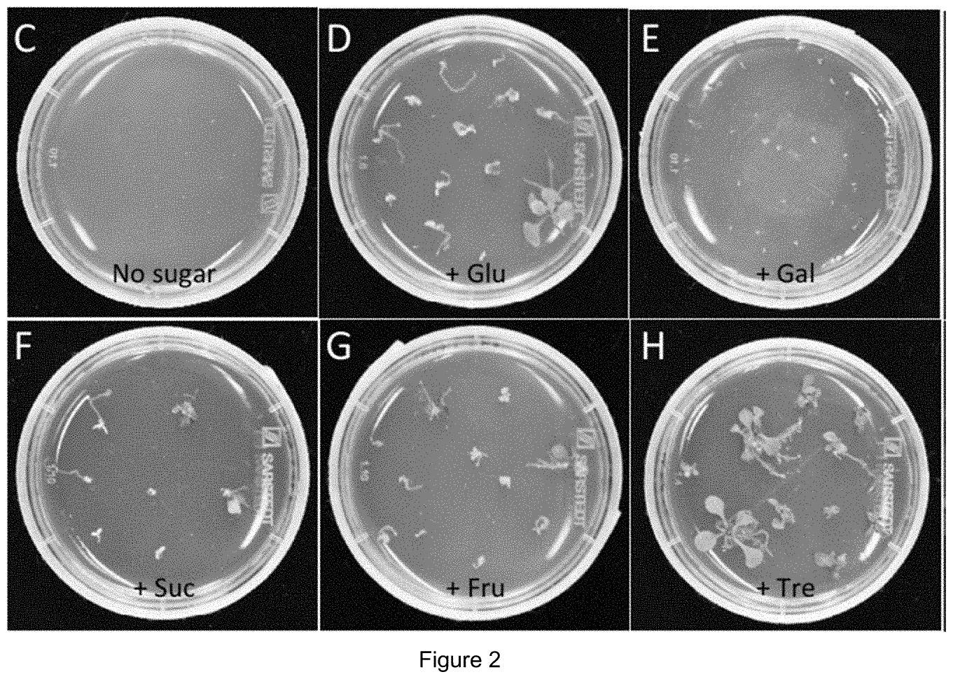

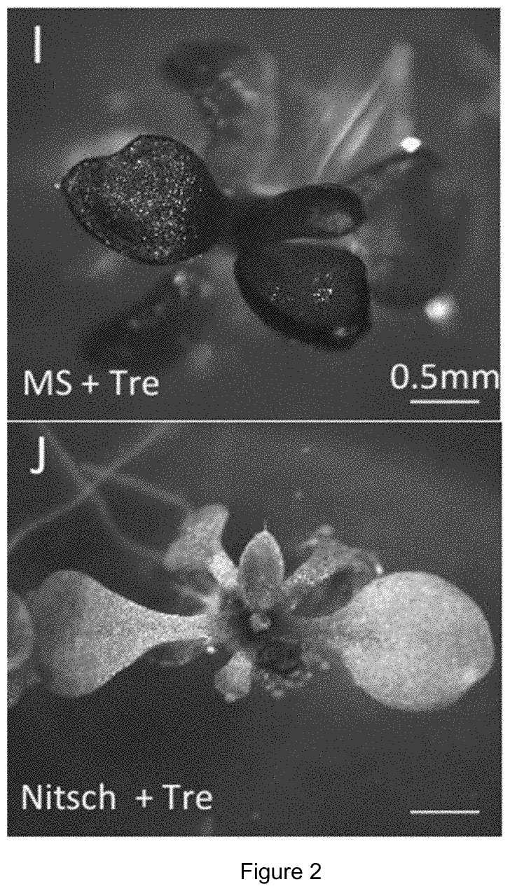

Experimental data as presented herein indicates that the survival rate of hybrid plant embryos is particularly increased when culturing the hybrid plant embryos in a culture medium comprising trehalose, and/or the derivative thereof, at a concentration of from 0.5% (w/v) up to 10% (w/v). The most optimal concentration may differ slightly from hybrid plant embryo to hybrid plant embryo as shown in the experimental data for triploid Arabidopsis thaliana embryo versus Capsella rubella×Capsella grandiflora embryo (FIGS. 1B and 2B).

The most optimal concentration of trehalose, and/or the derivative thereof, in the culture medium can be determined for a particular hybrid plant embryo by culturing such hybrid plant embryos in culture media supplemented with different concentrations of the trehalose, and/or the derivative thereof, and then determining the survival rate of the hybrid plant embryos as disclosed in the Example section. The concentration of trehalose, and/or the derivative thereof, resulting in the highest survival rate is then preferably selected for the particular hybrid plant embryo.

In an embodiment, culturing the hybrid plant embryo comprises culturing the hybrid plant embryo in the culture medium comprising trehalose, and/or the derivative thereof, until the hybrid plant embryo develops a root.

In this embodiment, the hybrid plant embryo is cultured in the culture medium supplemented with trehalose, and/or the derivative thereof, until the embryo develops a roots, i.e., has grown and developed into a plantling comprising a root or a root system. In a particular embodiment, the embryo has also developed a leaf or leaves. Hence, in a particular embodiment, hybrid plant embryos are kept in the culture medium supplemented with trehalose, and/or the derivative thereof, until formation of true roots, and preferably leaves. A this point, the resulting plantling can be transferred into soil or another plant substrate for further growth.

In an embodiment, it may be preferred to change culture medium during culturing of the hybrid plant embryo. For instance, the hybrid plant embryo could first be cultured in a first or initial culture medium for a given period of time and is then transferred for further growth in a second, different culture medium. The different culture media could then be adapted for the different developmental or embryogenesis stages of the hybrid plant embryo. For instance, the first culture medium could be adapted for growth of hybrid plant embryos in the heart embryogenesis stage or the torpedo embryogenesis stage. However, continuous culturing of the hybrid plant embryo into a plantling in this first culture medium may cause negative effects to the hybrid plant embryo, such as vitrification. In such a case, it may be preferred to transfer the hybrid plant embryo from the first culture medium into a second, different culture medium that suppresses or inhibits the negative effects.

In such an approach, the first culture medium could comprise trehalose, and/or the derivative thereof. Alternatively, the second culture medium comprises trehalose, and/or the derivative thereof, or both the first culture medium and the second culture medium comprise trehalose, and/or the derivative thereof. It is currently preferred if at least the first culture medium comprises trehalose, and/or the derivative thereof.

Hence, in a particular embodiment culturing the hybrid plant embryo comprises culturing the hybrid plant embryo in a first culture medium comprising trehalose, and/or the derivative thereof. This particular embodiment also comprises transferring the hybrid plant embryo to a second culture medium comprising trehalose, and/or the derivative thereof, and culturing the hybrid plant embryo in the second culture medium comprising trehalose, and/or the derivative thereof.

If both the first and second culture media comprise trehalose, and/or the derivative thereof, the two culture media may comprise the same concentration of trehalose, and/or the derivative thereof, or different concentrations of trehalose, and/or the derivative thereof. In the latter case, the first culture medium could comprise a higher concentration of trehalose, and/or the derivative thereof, as compared to the second culture medium, or the first culture medium comprises a lower concentration of trehalose, and/or the derivative thereof, as compared to the second culture medium. In a particular embodiment, the second culture medium comprises a lower concentration of trehalose, and/or the derivative thereof, than the first culture medium. For instance, the concentration of trehalose, and/or the derivative thereof, in the second culture medium could be equal to 90%, 80%, 70%, 60%, 50%, 40%, 30%, 20% or 10% of the concentration of trehalose, and/or the derivative thereof, in the first culture medium. A particular example is to have half the concentration of trehalose, and/or the derivative thereof, in the second culture medium as compared to the first culture medium.

In the embodiments of different culture media, the hybrid plant embryo is preferably cultured in the second culture medium comprising trehalose, and/or the derivative thereof, until the hybrid plant embryo develops a root.

Non-limiting, but illustrative, examples of culture media that can be used according to the embodiments include Murashige & Skoog (MS) medium (Murashige & Skoog 1962), Nitsch medium (Nitsch & Nitsch 1969), Gamborg B5 medium (Gamborg et al. 1968), White medium (White 1964), Eriksson medium (Eriksson 1965), H medium (Bourgin & Nitsch 1967), Miller (M) medium (Miller 1963), modified Miller (MM) medium (Chu et al., 1975), Keller (K) medium (Keller & Guillard 1985, Keller et al. 1987), NT medium (Nagata and Takebe, 1971), Anderson's Rhododendron (Anderson 1980), Chée & Pool medium (Chée and Pool, 1987), De Greef & Jacobs medium (De Greef and Jacobs, 1979), DKW/Juglans medium (Driver et al. 1984) and Kao & Michayluk medium (Kao et al. 1973). In fact, the present invention can be used in connection with any culture medium traditionally used for culturing hybrid plant embryos.

Another aspect of the invention relates to use of trehalose, and/or a derivative thereof, as a supplement to a culture medium for in vitro embryo rescue. In this aspect, a hybrid plant embryo is cultured in the culture medium supplemented with trehalose, and/or the derivative thereof.

The use of trehalose, and/or the derivative thereof, as a supplement to a culture medium for in vitro embryo rescue significantly increases the survival rate of the hybrid plant embryo as compared to culturing hybrid plant embryos in a culture medium lacking trehalose, and/or the derivative thereof.

A further aspect of the invention relates to an in vitro embryo rescue culturing system. The system comprises a culture vessel comprising a culture medium comprising trehalose, and/or a derivative thereof. The system also comprises a hybrid plant embryo present in the culture medium comprising trehalose and/or the derivative thereof.

The culture vessel could be any vessel or container used for culturing hybrid plant embryos. Non-limiting, but illustrative, examples of such culture vessels include Petri dishes, cell culture plates, flasks and plant culture bottles.

In an embodiment, the culture medium comprises trehalose, and/or the derivative thereof, at a concentration of from 0.5% (w/v) up to 10% (w/v). In a particular embodiment, the culture medium in the culture vessel comprises trehalose, and/or the derivative thereof, at a concentration of from 1% (w/v) up to 8% (w/v), and more preferably at a concentration of from 1.5% (w/v) up to 6% (w/v).

In an embodiment, the hybrid plant embryo is an interploidy or interspecies hybrid plant embryo.

The culture medium in the culture vessel can advantageously be selected among the above described examples of culture media.

### EXAMPLES

### Example 1

The following three plant species were employed in the present Example: Arabidopsis thaliana (Col-0), Capsella rubella (Cr48.21) and Capsella grandiflora (Cg88.14).

### Materials & Methods

Seeds of Arabidopsis and Capsella were surface sterilized (5% sodium hypochlorite, 0.05% Triton X-100) for 10 min, washed three times in sterile ddH₂O and then plated on half Murashige and Skoog (MS) medium, i.e., MS medium as defined below but half the concentration of the included ingredients. After stratification for 3 day at 4° C., plants were grown in a growth room under long-day photoperiod (16 hours light and 8 hours darkness) at 22° C. Eight-day-old seedlings were transferred to soil and plants were grown in a growth room at 60% humidity and daily cycles of 16 hours light at 22° C. and 8 hours darkness at 18° C. For crosses, designated female flower buds were emasculated, and the pistils hand-pollinated at 2 days after emasculation.

### Solutions and Culture Media

70% Ethanol;
Sterilizing solution (5% sodium hypochlorite+0.01% (v/v) of Triton X-100 or TWEEN® 20);
Autoclaved water;
Murashige & Skoog (MS) medium (Murashige and Skoog 1962): MS basal salt mixture 4.4 g/L, 0.7% plant agar, pH 5.8 and vitamins (glycine 2 mg/I, myo-inositol 100 mg/I, nicotinic acid 0.5 mg/I, pyridoxine HCl 0.5 mg/I and thiamine HCl 0.1 mg/I);
Nitsch medium (Nitsch & Nitsch 1969): basal salt mixture 2.1 g/L, 0.7% plant agar, pH 5.8 and vitamins (biotin 0.05 mg/I, folic acid 0.5 mg/I, glycine 2 mg/I, myo-inositol 100 mg/I, nicotinic acid 5 mg/I, pyridoxine HCl 0.5 mg/I and thiamine HCl 0.5 mg/I);

### Results

### The Rescue Culture of Immature Embryos

Triploid Arabidopsis seeds were derived from crossing diploid Arabidopsis females with tetraploid Arabidopsis pollen donors. Interspecific hybrid Capsella seeds were derived from crossing Capsella rubella females with Capsella grandiflora pollen donors. Hybrid embryos were isolated at the torpedo stage, at around 8 to 9 days after pollination (DAP) for Arabidopsis and 9 to 10 DAP for Capsella.

Under a sterile hood, the siliques of the crosses were incubated for 30 s in 70% ethanol, 30 s in sterilizing solution and 30 s in sterile water. The siliques were placed on MS medium supplemented with 3% (w/v) trehalose overlaid with a filter paper and several drops of sterile liquid MS medium were added on the silique. Under a dissection microscope, the silique was opened with fine needles, and all seeds were transferred on the wet filter paper. The embryos were gently dissected and placed on the filter paper. During the dissection, the embryos and seeds should not become desiccated. The plates were sealed with Millipore tape and incubated under long-day photoperiod (16 hours light and 8 hours darkness) at 22° C.

### Supporting Culture for Embryo Growth

After 5-7 days culture on MS medium with trehalose for Arabidopsis or 7-10 days for Capsella, viable embryos could be recognized by their green color and increased size that corresponded to the size of embryos at the bent cotyledon stage (FIG. 1J). Viable embryos were then transferred to Nitsch medium containing lower concentrations of trehalose, 1.5% (w/v) for Arabidopsis, 3% (w/v) for Capsella. Embryos were transferred from MS medium to Nitsch medium after two weeks. Transfer to Nitsch medium reduced vitrification, which is a frequently occurring phenomenon in tissue-cultured plants causing excessive hydration of tissues and reducing survival rate. Continued growth on MS medium for 3 weeks caused vitrification in Arabidopsis and Capsella seedlings (FIGS. 1L, 2I), but this phenomenon was suppressed by transfer to Nitsch medium (FIGS. 1M, 2J). After about two weeks of incubation, the initiation of roots could be observed (FIG. 1K) and the plantlets could be transferred to soil and continued to grow under long-day photoperiod (16 h light/8 h dark) at 22° C. in a growth room.

Arabidopsis thaliana

Triploid Arabidopsis thaliana (Col-0) embryos at the torpedo stage were dissected under a stereomicroscope under sterile conditions on a laminar flow bench. The isolated embryos were cultured in vitro on MS medium supplemented with different sugars. The addition of 3% (w/v) glucose, 3% (w/v) galactose, 3% (w/v) sucrose or 3% (w/v) fructose did not improve embryo survival rate (FIG. 1A), which was about 10% or less. In contrast, the addition of 3% (w/v, 87.6 mM) trehalose could rescue over 70% of hybrid embryos. The majority of triploid embryos on MS medium supplemented with glucose, galactose, sucrose and fructose turned from light green to white and growth was arrested (FIGS. 1C-1H). We tested which concentration of trehalose conferred the highest survival rates (0%, 1%, 1.5%, 2%, 3%, 6%, 12% (w/v)). Isolated embryos had the highest survival rates (over 90%) when cultured on plates containing 2% (w/v) trehalose (FIG. 1B). Surviving embryos were kept in a light culture room for two weeks until they formed true leaves and roots and the plants were then transferred into soil after another two weeks of culture.

### Capsella

To rescue interspecific hybrids of Capsella, the protocol was the same as above for A. thaliana but with minor changes for the trehalose concentration. Hybrid embryos of Capsella (Capsella rubella×Capsella grandiflora) were dissected at the torpedo stage under a stereomicroscope under sterile conditions on a laminar flow bench. The isolated embryos were cultured on MS medium supplemented with 3% (w/v, 87.6 mM) trehalose. Like for Arabidopsis triploid embryos, sugars like glucose, galactose, sucrose and fructose did not improve embryo survival (FIG. 2A). Addition of trehalose could rescue 80% of the hybrid embryos. The average survival rate on other sugars was around 10% (FIG. 2A).

The majority of triploid embryos on MS medium supplemented with glucose, galactose, sucrose and fructose turned from light green to white and growth was arrested (FIGS. 2C-2H). Isolated embryos had the highest survival rate (over 90%) when cultured on plates containing 6% trehalose (FIG. 2B). Embryos were kept in a light culture room for two weeks until the formation of true leaves and roots and the plants were then transferred into soil after another two weeks (FIG. 2B).

### Example 2

### Results

### Rescue of Triploid Rice Embryos

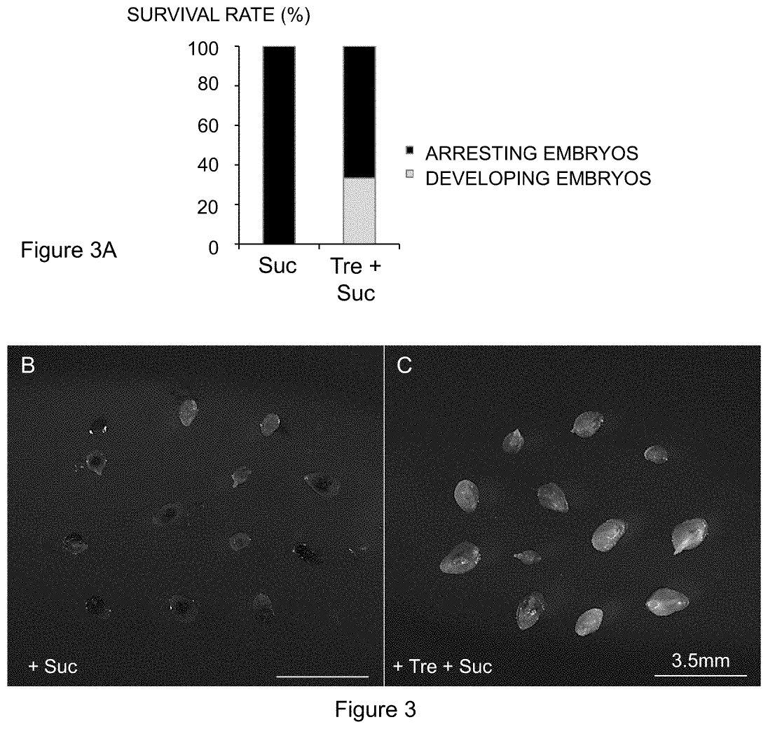

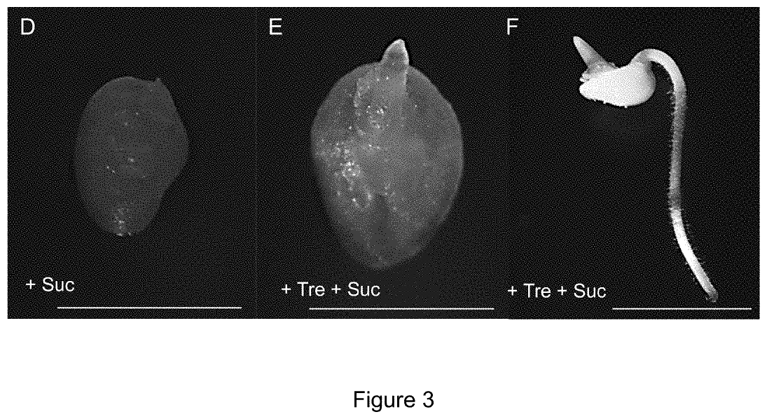

To obtain triploids of cultivated rice (Oryza sativa L. cv. Suitou nourin 8), diploid plants (JP9838) were emasculated and pollinated with pollen from tetraploid plants (JP7525). Triploid hybrid embryos were dissected under sterile conditions at 10-12 days after pollination (DAP) under a stereomicroscope. The isolated embryos were cultured in vitro on MS medium supplemented with 1.5% (w/v, 43.8 mM) trehalose together with sucrose (1.5%) and compared to MS medium supplemented with only sucrose (3%). Around 30% of rice triploid embryos grown on medium containing trehalose (1.5%) survived after one week of culture, as visible by the embryos turning light green (FIGS. 3A and 3C). In contrast, none of the embryos grown on medium containing only sucrose (3%) survived; they arrested growth and turned dark brown (FIGS. 3A and 3B). The viable triploid embryos were transferred to MS medium containing lower concentrations of trehalose (0.75% w/v) together with 2.25% (w/v) sucrose for two weeks, until the initiation of roots could be observed (FIG. 3F). The plantlets were transferred to soil and their ploidy levels were validated by flow cytometry.

### Materials & Methods

### Plant Materials and Growth Conditions

Cultivated rice (Oryza sativa L. cv. Suitou nourin 8, diploid, JP9838, and cv. Suitou nourin 8, tetraploid, JP7525) seeds were obtained from the National Institute of Agrobiological Sciences in Japan (http://www.gene.affrc.go.jp). Rice seeds were grown on soil and cultivated in a phytotron. Cultivating conditions were 14 h light and 10 h dark at 30° C./21° C. with a constant humidity of 80% and light intensity of 400 μmol per m⁻² s⁻¹. For crossing, designated diploid plants were emasculated and pollination was performed immediately after emasculation.

### The Rescue Culture of Immature Embryos

Under a sterile hood, immature triploid rice seeds at 10-12 DAP were dehusked using a pair of tweezers and sterilized for 2 min in 70% ethanol, followed by 15 min in 5% sodium hypochlorite with gentle mixing, 2 min in sterile water. The seeds were placed on MS medium supplemented with 1.5% (w/v) trehalose together with 1.5% (w/v) sucrose overlaid with a filter paper. Under a dissection microscope, the seed coat was removed with fine needles, and dissected embryos were transferred on the wet filter paper. During the dissection, the embryos should not become desiccated. The plates were sealed with Millipore tape and incubated under long-day photoperiod (16 hours light and 8 hours darkness) at 22° C. After 5-7 days of culture on MS medium with trehalose, viable embryos could be recognized by their light green color and increased size (FIG. 3E), compared to aborted embryos with dark brown color (FIG. 3D). Viable embryos were then transferred to MS medium containing lower concentrations of trehalose, 0.75% (w/v) together with 2.25% (w/v) sucrose. After about two weeks of incubation, the initiation of roots could be observed (FIG. 3F) and the plantlets could be transferred to soil and grows in a phytotron.

The embodiments described above are to be understood as a few illustrative examples of the present invention. It will be understood by those skilled in the art that various modifications, combinations and changes may be made to the embodiments without departing from the scope of the present invention. In particular, different part solutions in the different embodiments can be combined in other configurations, where technically possible. The scope of the present invention is, however, defined by the appended claims.

### REFERENCES

Anderson, “Tissue culture propagation of red and black raspberries, Rubus idaeus and R. occidentalis” Symposium on Breeding and Machine Harvesting of Rubus (1980), article no. 112, pages 13-20
Bourgin and Nitsch, “Production of haploid Nicotiana from excised stamens” Ann Physiol Veg (1967) 9: 377-382
Chee and Pool, “Improved inorganic media constituents for in vitro shoot multiplication of Vitis” Scientia Horticulturae (1987) 32(1-2: 85-95
Chu, et al., “Establishment of an efficient medium for anther culture of rice through comparative experiments on the nitrogen sources” Sci Sin (1975) 18:659-668
De Greef and Jacobs, “In vitro culture of the sugarbeet: description of a cell line with high regeneration capacity” Plant Science Letters (1979) 17(1): 55-61

Driver and Kuniyuki, “In vitro propagation of Paradox walnut rootstock” HortScience (1984) 19(4): 507-509
Eriksson, “Studies on the growth requirements and growth measurements of cell cultures of Haplopappus gracilis” Physiologia Plantarum (1965) 18(4): 976-993
Esen and Soost, “Seed development in Citrus with special reference to 2××4×crosses” American Journal of Botany (1973) 60(5): 448-462
Gadwal, et al., “Interspecific hybrids in Abelmoschus through ovule and embryo culture” Indian Journal of Genetics and Plant Breeding (1968) 28(3): 269
Gamborg et al., “Nutrient requirements of suspension cultures of soybean root cells” Experimental cell research (1968) 50(1): 151-158
Kao, et al. “Effects of sugars and inorganic salts on cell regeneration and sustained division in plant protoplasts” Colloques Internationaux du Centre National de la Rechsci Protoplastes et Fusion de Cell Somatiques Vegetates (1973)
Keller and Guillard, “Factors significant to marine diatom culture” pp 113-116 in Anderson, White and Baden (eds.) Toxic Dinoflagellates, (1985)
Keller, et al., “Media for the culture of oceanic ultraphytoplankton” J. Phycol. (1987) 23: 633-638
Köhler, et al., “The impact of the triploid block on the origin and evolution of polyploid plants” Trends in Genetics (2010) 26(3): 142-148
Kradolfer, et al., “Increased maternal genome dosage bypasses the requirement of the FIS polycomb repressive complex 2 in Arabidopsis seed development” PLoS genetics (2013) 9(1): e1003163
Marks, “The origin and significance of intraspecific polyploidy: experimental evidence from Solanum chacoense” Evolution (1966) 20(4): 552-557
Miller, “Kinetin and kinetin-like compounds.” Modern Methods of Plant Analysis/Moderne Methoden der Pflanzenanalyse, Springer, Berlin, Heidelberg (1963) 194-202
Murashige and Skoog, “A Revised Medium for Rapid Growth and Bio Assays with Tobacco Tissue Cultures” Physiol. Plant (1962) 15: 473
Nagata and Takebe, “Plating of isolated tobacco mesophyll protoplasts on agar medium.” Planta (1971) 99(1): 12-20
Nitsch J. P. and Nitsch C., Haploid plants from pollen grains, Science 169, 85 (1969).
Rebernig, et al., “Non-reciprocal interspecies hybridization barriers in the Capsella genus are established in the endosperm” PLoS genetics (2015) 11(6): e1005295
Sahijram, et al., “Hybrid embryo rescue: a non-conventional breeding strategy in horticultural crops, The Journal of Horticultural Science and Biotechnology 2013) 8(1): 1-20
Sharma, “How wide can a wide cross be?” Euphytica (1995) 82(1): 43-64
White, “The cultivation of animal and plant cells” Soil Science (1964) 97(1): 74.
Zhang, et al., “Parental Genome Imbalance Causes Post-Zygotic Seed Lethality and Deregulates Imprinting in Rice” Rice (2016) 9(1): 43

1. A method of in vitro embryo rescue comprising culturing a hybrid plant embryo in a culture medium comprising trehalose, and/or a derivative thereof.

2. The method according to claim 1, further comprising isolating the hybrid plant embryo from a seed of a plant, wherein culturing the hybrid plant embryo comprises culturing the isolated hybrid plant embryo in the culture medium comprising trehalose, and/or the derivative thereof.

3. The method according to claim 2, wherein isolating the hybrid plant embryo comprises dissecting the hybrid plant embryo from the seed in a culture medium comprising trehalose, and/or the derivative thereof.

4. The method according to claim 1, wherein culturing the hybrid plant embryo comprises culturing a hybrid plant embryo at a heart embryogenesis stage or a torpedo embryogenesis stage.

5. The method according to claim 1, wherein culturing the hybrid plant embryo comprises culturing a hybrid plant zygotic embryo formed by fusion of male and female gametes in the culture medium comprising trehalose, and/or the derivative thereof.

6. The method according to claim 1, wherein culturing the hybrid plant embryo comprises culturing an interploidy or interspecies hybrid plant embryo in the culture medium comprising trehalose, and/or the derivative thereof.

7. The method according to claim 1, wherein culturing the hybrid plant embryo comprises culturing the hybrid plant embryo in the culture medium comprising trehalose, and/or the derivative thereof, at a concentration of from 0.5% (w/v) up to 10% (w/v).

8. The method according to claim 1, wherein culturing the hybrid plant embryo comprises culturing the hybrid plant embryo in the culture medium comprising trehalose, and/or the derivative thereof, until the hybrid plant embryo develops a root.

9. The method according to claim 1, wherein culturing the hybrid plant embryo comprises:
culturing the hybrid plant embryo in a first culture medium comprising trehalose, and/or the derivative thereof;
transferring the hybrid plant embryo to a second culture medium comprising trehalose, and/or the derivative thereof; and
culturing the hybrid plant embryo in the second culture medium comprising trehalose, and/or the derivative thereof.

10. The method according to claim 9, wherein the second culture medium comprises a lower concentration of trehalose, and/or the derivative thereof, than the first culture medium.

11. The method according to claim 10, wherein the second culture medium comprises half the concentration of trehalose, and/or the derivative thereof, of the first culture medium.

12. The method according to claim 9, wherein culturing the hybrid plant in the second culture medium comprises culturing the hybrid plant embryo in the second culture medium comprising trehalose, and/or the derivative thereof, until the hybrid plant embryo develops a root.

13. (canceled)

14. An in vitro embryo rescue culturing system comprising:
a culture vessel comprising a culture medium comprising trehalose, and/or a derivative thereof; and
a hybrid plant embryo present in the culture medium comprising trehalose, and/or the derivative thereof.

15. The system according to claim 14, wherein the culture medium comprises trehalose, and/or the derivative thereof, at a concentration of from 0.5% (w/v) up to 10% (w/v).

16. The system according to claim 14, wherein the hybrid plant embryo is an interploidy or interspecies hybrid plant embryo.

17. The method according to claim 1, wherein culturing the hybrid plant embryo comprises culturing a hybrid plant embryo at a torpedo embryogenesis stage.

18. The method according to claim 1, wherein culturing the hybrid plant embryo comprises culturing the hybrid plant embryo in the culture medium comprising trehalose, and/or the derivative thereof, at a concentration of from 1% (w/v) up to 8% (w/v).

19. The method according to claim 1, wherein culturing the hybrid plant embryo comprises culturing the hybrid plant embryo in the culture medium comprising trehalose, and/or the derivative thereof, at a concentration of from 1.5% (w/v) up to 6% (w/v).

20. The system according to claim 14, wherein the culture medium comprises trehalose, and/or the derivative thereof, at a concentration of from 1% (w/v) up to 8% (w/v).

21. The system according to claim 14, wherein the culture medium comprises trehalose, and/or the derivative thereof, at a concentration of from 1.5% (w/v) up to 6% (w/v).
